(19) United States
(12) Patent Application Publication
(10) Pub. No.: US 2009/0163927 A1
(43) Pub. Date: Jun. 25, 2009

(54) DELIVERY TOOLS AND METHODS FOR INTRAVASCULAR IMPLANTABLE DEVICES

(76) Inventors: Terrance Ransbury, Chapel Hill, NC (US); Kevin Holbrook, Chapel Hill, NC (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/327,785

(22) Filed: Dec. 3, 2008

Related U.S. Application Data
(63) Continuation of application No. 12/327,808, filed on Dec. 3, 2008.
(60) Provisional application No. 61/005,354, filed on Dec. 3, 2007.

(51) Int. Cl. A61B 17/00; A61N 1/02

(52) U.S. Cl. 606/129

(57) ABSTRACT

Improved methods and apparatuses for positioning intravascular implantable device (IID) in a patient's vasculature utilize a device delivery system having an elongated flexible body. A handle can be operably connected to the proximal end and a grasper mechanism can be positioned at the distal end of the device delivery system. The grasper mechanism can be configured to releasably grasp the IID by closing a releasable honda around the IID and can be selectively controllable with the handle.

### RELATED APPLICATIONS

The present invention claims priority to U.S. Provisional Patent Application No. 61/005,354, entitled “Implantation Methods, Systems and Tools for Intravascular Implantable Devices,” filed Dec. 3, 2007 which is hereby incorporated by reference in its entirety.

### FIELD OF THE INVENTION

The present invention generally relates to devices, systems and methods for diagnosing and treating patients. In particular, the invention relates to delivery tools and methods for intravascular implantable devices.

### BACKGROUND OF THE INVENTION

Implantable devices that provide long-term active therapies such as pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high energy electrical stimuli to slow or reset the heart rate in the event a ventricular tachycardia (VT) or ventricular fibrillation (VF) is detected. Another type of implantable device detects an atrial fibrillation (AF) episode and delivers an electrical stimuli to the atria to restore electrical coordination between the upper and lower chambers of the heart. The current generation for all of these implantable cardiac rhythm management (CRM) devices are typically can-shaped devices implanted under the skin that deliver electrical stimuli via leads that are implanted in the heart via the patient's vascular system. Conventional pacemakers and ICDs are implanted subcutaneously, typically in the pectoral region.

Most implantable pulse generators use conventional leads in the form of elongated, floppy lead bodies that insulate, seal and protect one or more conductors which transmit electrical pulses between the pulse generator and one or more electrodes on the distal portion of the lead. The one or more intravascular leads associated with a conventional pulse generator are typically not integrated with the device; instead, a header is provided on the device for connecting the one or more leads to the device. The lead tip is affixed in, on, or near the heart, depending on the desired treatment. To implant the one or more intravascular leads for a conventional pulse generator, a stylet inserted into an open lumen at the proximal end of the lead is used to navigate the lead into the subclavian vein, through the superior vena cava, and on to the heart. Implantation of the device typically follows implantation of the lead. The stylet is removed from the lead, the lead is connected to the device, and the device is then secured in the patient.

Next generation long-term active implantable devices may take the form of elongated intravascular devices that are implanted within the patient's vascular system, instead of under the skin. Examples of these intravascular implantable devices (IIDs) are described in, for example, U.S. Pat. No. 7,082,336 and U.S. Published Patent Application Nos. 2005/0043765A1, 2005/0228471A1 and 2006/0217779A1. These devices contain electric circuitry and/or electronic components that must be hermetically sealed to prevent damage to the electronic components and the release of contaminants into the bloodstream. Due to the length of these implantable devices, which in some cases can be approximately 10-60 cm in length, the devices must be flexible enough to move through the vasculature while being sufficiently rigid to protect the internal components.

As described in U.S. Pat. No. 7,082,336, techniques for implanting such an intravascular implantable device generally begin by obtaining access to the vasculature of the patient through a puncture made in a vessel, such as one of the femoral veins in the leg. As described in U.S. Pat. No. 7,082,336, an over-the-wire implant technique can be used for implanting an IID. After an incision is made in a vessel, an introducer is inserted into the incision to keep the vein open during the procedure. A guide catheter is inserted through the introducer and a guide wire is directed into a vessel superior to the heart. The guide catheter is removed, leaving the guide wire in place. The distal portion of the device can include a passage for the wire or the device body can include a passage for the wire. The device is then inserted onto the guide wire, and the device is manually inserted into the vasculature until the proximal end of the device reaches the introducer. A pusher in the form of a mandrel that is detachably coupled to the proximal end of the elongated device can be utilized to push the device into a position where the device could be anchored using an anchoring system.

While such a pushing arrangement permits positive control of the proximal end of the device, the mechanical nature of the mandrel coupling can complicate the construction and implantation of the device and can present challenges with respect to issues of effective hermetic sealing and potential thrombosis formation near this region of the device, as well as complicating lead placement on the proximal end of the device. In addition, next-generation IIDs may include a lead integrally connected to the proximal end of the IID, preventing the use of a mandrel pusher.

While intravascular implantable devices represent a significant improvement over conventional long-term active implantable devices that are implanted subcutaneously, there are opportunities to improve and refine the implantation techniques, system and tools for implanting such intravascular implantable devices. It would be desirable to provide improved methods, systems and tools for implanting such intravascular implantable devices that can simplify the implantation of these devices so as improve the effectiveness and ease of the procedure.

### SUMMARY OF THE INVENTION

Improved methods and apparatuses for positioning intravascular implantable device (IID) in a patient's vasculature utilize a device delivery system having an elongated flexible body. A handle can be operably connected to the proximal end and a grasper mechanism can be positioned at the distal end of the device delivery system. The grasper mechanism can be configured to releasably grasp the IID by closing a releasable honda around the IID and can be selectively controllable with the handle.

In one embodiment, a device delivery system can have an elongated flexible body adapted to be implanted into a patient's vasculature. The body can have a proximal end and a distal end and can include a flexible wire. A handle can be operably connected to the proximal end of the device body and a grasper mechanism can be connected to the distal end of the flexible wire. The grasper mechanism can be configured to releasably grasp an IID by closing a releasable honda that is selectively controllable with the handle around the IID. In one embodiment, the releasable honda can be closed around the IID by inserting an end portion of the releasable honda into a collar section located at the distal end of the device body. In another embodiment, the releasable honda can be closed around the IID by connected a stylet projecting from device body with the releasable honda. The releasable honda can be configured to release its grasp on the IID via operation of the handle. Device delivery system allows for positive control of the IID.

To position an IID in a patient's body utilizing a device delivery system, first an incision can be formed in the vasculature of the patient. An introducer sheath can be used to maintain the opening formed by the incision. The device delivery system can be used to grasp the IID by closing a releasable honda around the IID. The IID can then be guided through the patient's vasculature by controlling the device delivery system. Once the IID is in a desired location, the handle of the device delivery system can be used to release the IID. In one embodiment, at least a portion of the device delivery system can then be withdrawn from the patient's vasculature. In another embodiment, device delivery system can retain position control until after a lead associated with the IID is implanted.

The above summary of the various embodiments of the invention is not intended to describe each illustrated embodiment or every implementation of the invention. This summary represents a simplified overview of certain aspects of the invention to facilitate a basic understanding of the invention and is not intended to identify key or critical elements of the invention or delineate the scope of the invention.

### BRIEF DESCRIPTION OF THE DRAWINGS

The invention may be more completely understood in consideration of the following detailed description of various embodiments of the invention in connection with the accompanying drawings, in which:

FIG. 1 is a perspective illustration depicting human cardiac anatomy.

FIG. 2 is a schematic generally depicting components of an intravascular electrophysiological system according to one aspect of the present invention.

FIG. 3 is a schematic of one embodiment of an intravascular implantable device according to the present invention.

FIG. 4A is a perspective view of a device delivery system according to one aspect of the present invention.

FIG. 4B is an exploded perspective view of the device delivery system of FIG. 4A.

FIG. 4C is a top plan view of the device delivery system of FIG. 4A.

FIG. 4D is a side plan view of the device delivery system of FIG. 4A.

FIG. 4E is an end plan view of the device delivery system of FIG. 4A.

FIG. 5A is a perspective view of a device body of a device delivery system according to one aspect of the present invention.

FIG. 5B is a top plan view of the device body of FIG. 5A, depicted without a sheath for clarity.

FIG. 5C is a side plan view of the device body of FIG. 5B.

FIG. 5D is a top plan view of the device body of FIG. 5A.

FIG. 5E is a side plan view of the device body of FIG. 5A.

FIG. 6 is a view of an intravascular implantable device being guided into a patient's body with a device delivery system according to one aspect of the present invention.

FIG. 7A is a perspective view of a grasper mechanism according to one aspect of the present invention in a free state.

FIG. 7B is a perspective view of the grasper mechanism of FIG. 7A in an engaged state.

FIG. 8A is a side plan view of a device delivery system grasping an intravascular implantable device according to one aspect of the present invention.

FIG. 8B is a perspective view of the device delivery system grasping an intravascular implantable device of FIG. 8A.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

### Anatomy

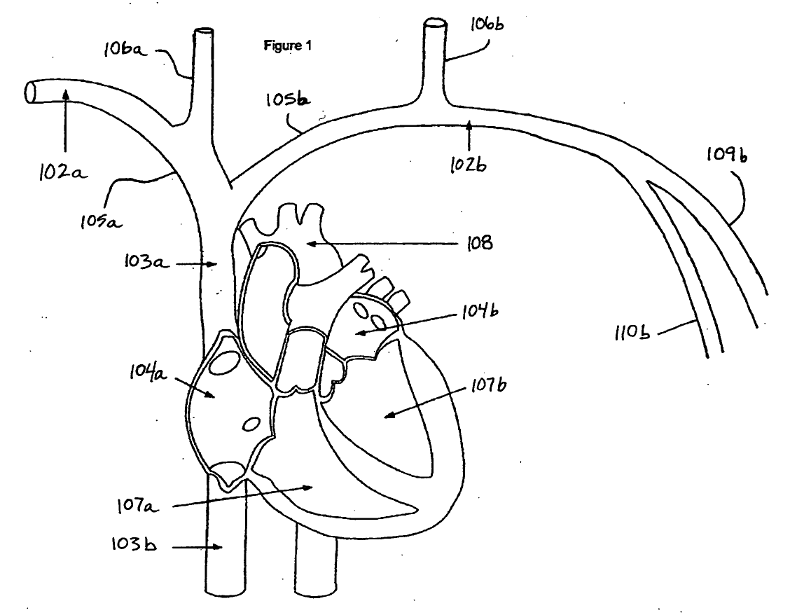

Referring to FIG. 1, the general cardiac anatomy of a human is depicted, including the heart and major vessels. The following anatomic locations are shown and identified by the listed reference numerals: Right Subclavian 102a, Left Subclavian 102b, Superior Vena Cava (SVC) 103a, Inferior Vena Cava (IVC) 103b, Right Atrium (RA) 104a, Left Atrium (LA) 104b, Right Innominate/Brachiocephalic Vein 105a, Left Innominate/Brachiocephalic Vein 105b, Right Internal Jugular Vein 106a, Left Internal Jugular Vein 106b, Right Ventricle (RV) 107a, Left Ventricle (LV) 107b, Aortic Arch 108, Descending Aorta 109, Right Cephalic Vein 109a (not shown in FIG. 1), Left Cephalic Vein 109b, Right Axillary Vein 110a (not shown in FIG. 1) and Left Axillary Vein 110b. Reference number 100 refers generally to vessels and/or vessel walls within the human body.

### The Kit

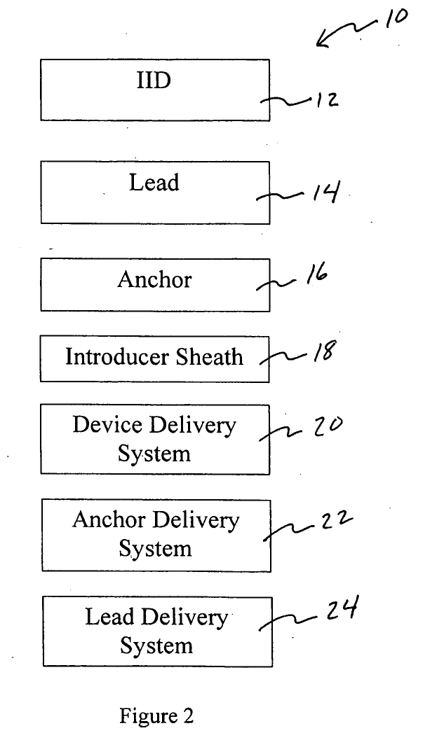

One configuration of the components of an electrophysiological treatment system 10 is depicted in FIG. 2. System 10 generally includes an intravascular implantable device (IID) 12 having a lead 14, the device being retained within a vessel by an anchor 16. An introducer sheath 18 is provided for implantation of system 10. A device delivery system 20 may be used to navigate the IID to the desired location. An anchor delivery system 22 can deliver anchor 16 to the desired location. Anchor delivery system 22 may also include a means for fixing or deploying anchor 16. A lead delivery system 24 is provided for maneuvering lead 14 to its desired location.

In one embodiment, instructions for implanting the system 10 in accordance with the various embodiments described herein in the form of printed or electronically, optically or magnetically stored information to be displayed, for example, are provided as part of a kit or assemblage of items prior to surgical implantation of the system 10. In another embodiment, instructions for implanting the system 10 in accordance with the various embodiments described herein are provided, for example, by a manufacturer or supplier of system 10, separately from providing the system 10, such as by way of information that is accessible using the Internet or by way of seminars, lectures, training sessions or the like.

### Structure of the Intravascular Implantable Device

Referring generally to FIGS. 3A-3B, an IID 200 according to one aspect of the present invention is depicted. In one embodiment, the IID 200 includes components known in the art to be necessary to carry out the system functions. For example, the IID 200 may include one or more pulse generators, including associated batteries, capacitors, microprocessors, and circuitry for generating electrophysiological pulses for defibrillation, cardioversion and/or pacing. The IID 200 also includes detection circuitry for detecting arrhythmias or other abnormal activity of the heart. The specific components to be provided in the device will depend upon the application for the device, and specifically whether the device is intended to perform defibrillation, cardioversion and/or pacing along with its sensing functions.

The IID 200 is proportioned to be passed into the vasculature and to be anchored within the patient's vasculature with minimal obstruction to blood flow. Suitable sites for the IID 200 may include, but are not limited to, the venous system using access through the right or left femoral vein or the subclavian or brachiocephalic veins, or the arterial system using access through one of the femoral arteries. Thus, the housing of the IID 200 preferably has a streamlined maximum cross sectional diameter which may be in the range of 3-15 mm or less, with a most preferred maximum cross-sectional diameter of 3-8 mm or less. The cross-sectional area of the device 200 in the transverse direction (i.e. transecting the longitudinal axis) should be as small as possible while still accommodating the required components. The cross-section of the device 200 (transecting the longitudinal axis) may have a circular cross-section, although other cross-sections including crescent, flattened, or elliptical cross-sections may also be used. It can be desirable to provide the device with a smooth continuous contour so as to avoid voids or recesses that could encourage thrombus formation on the device.

The lead 202 may be integrated with the device body 204, or attachable to the device body 204 in situ or prior to implantation, or the lead 202 may be integral with the device as an extension of the device itself. More than one lead 202 may be provided. Leads 202 may be included on the proximal end 206 of the device body 204, on the distal end 208 of the device body 204, generally on the device body, and/or any combination thereof. A lead 202 includes one or more defibrillation and/or pacing electrodes and may also be equipped to sense electrical activity of the heart. Monitoring of the heart's electrical activity can be needed to detect the onset of an arrhythmia. Activity sensed by the sensing electrode(s) is used by the device electronics to trigger delivery of a defibrillation shock. The lead 202 may be a conventional defibrillation/pacing lead, although alternative lead configurations may be desirable if warranted by the desired placement of the IID 200 and lead 202 within the body.

For leads 202 that are to be positioned within a chamber of the heart, the leads 202 may be the helical screw-in or tined variety for fixation to the cardiac tissue, and/or they may have steroid-eluding tips to facilitate tissue in-growth for fixation purposes. If a detachable tip is used, the lead tip 210 may be left within the chamber of the heart when the remainder of the lead 202 is removed, so as to prevent damage to the heart tissue as could occur upon extraction of the tined tip.

The leads 202 may include non-thrombogenic and/or non-proliferative surfaces or coatings, for example, the leads 202 may include a coating that is anti-thrombogenic (e.g. perfluorocarbon coatings applied using supercritical carbon dioxide) so as to prevent thrombus formation on the lead 202. It is also beneficial for the coating to have anti-proliferative properties so as to minimize endothelialization or cellular ingrowth, since minimizing growth into or onto the lead 202 will help minimize vascular trauma when the device is explanted. The coating may thus also be one which elutes anti-thrombogenic compositions (e.g. heparin sulfate) and/or compositions that inhibit cellular in-growth and/or immunosuppressive agents.

Thus, it should be appreciated that in this disclosure the term “lead” is used to mean an element that includes conductors and electrodes and that thus may be positioned somewhat remotely from the circuitry that energizes the electrodes. In other embodiments, leads may include elements that are simply extensions or tapers of the IID itself (such as the portion of the device at which electrodes are located) as well as more conventional intravascular leads.

In accordance with one embodiment of the present invention, the IID 200 can include at a distal end 208 an anchor attachment feature that allows the IID 200 to be disposed within the vasculature. An anchor detachment feature may be included so as to allow for removal of the IID 200 at a later date without damaging the vasculature by removing the anchor. An anchor zone may be disposed between the anchor attachment feature and the detachment feature for positioning the IID 200 between an anchor and the vasculature wall. In one embodiment, a telemetry antenna may be disposed axially along the distal end 208 proximate the anchor zone.

Additional disclosure pertaining to the structure and layout of intravascular implantable devices, as well as leads and anchors, can be found in U.S. Published Patent Application Nos. 2006/0217779, 2007/0265673, 2008/0147168, and 2008/0167702, the disclosures of which are hereby incorporated by reference.

### Device Delivery

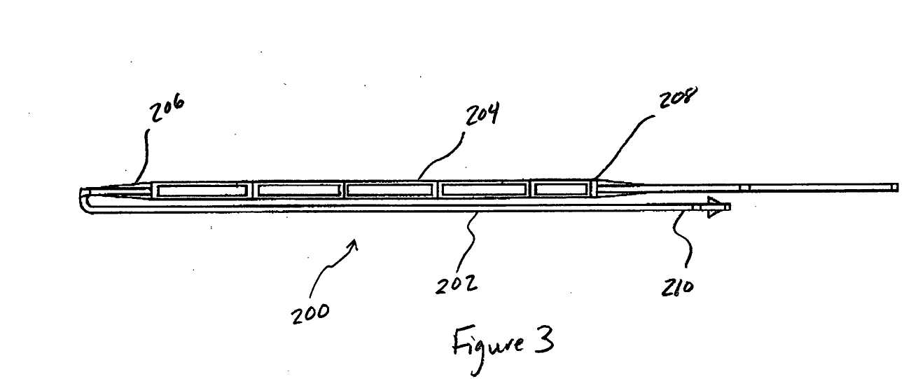

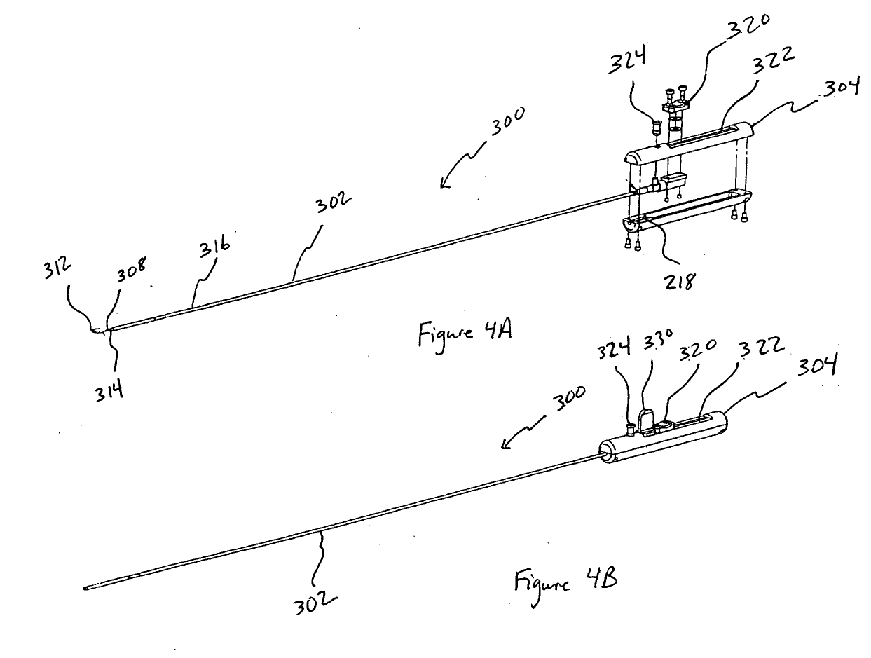

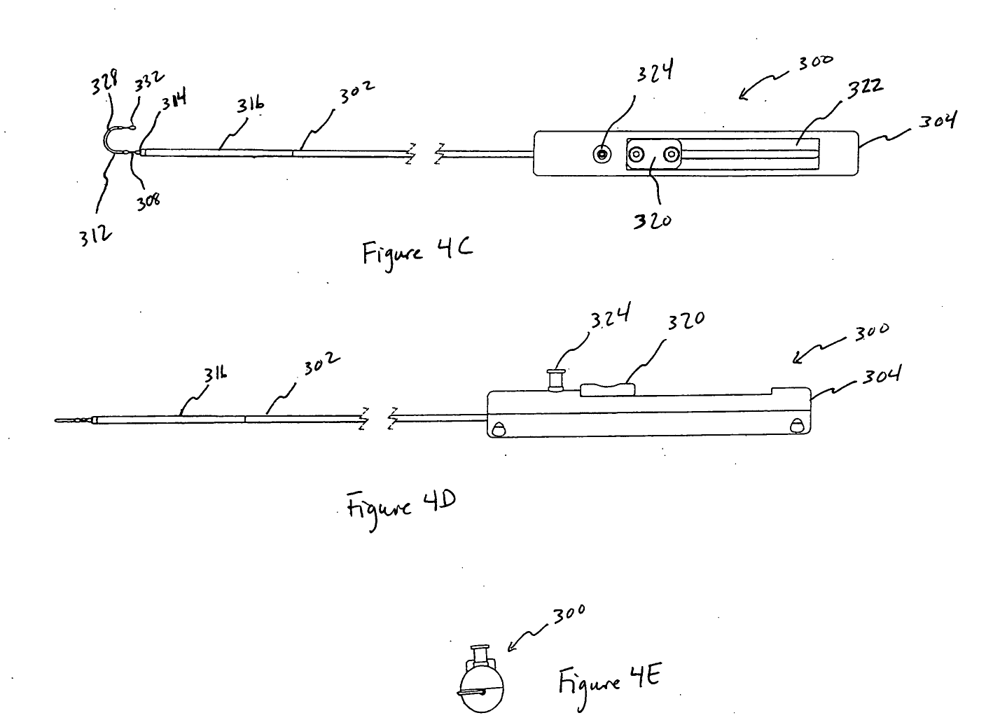

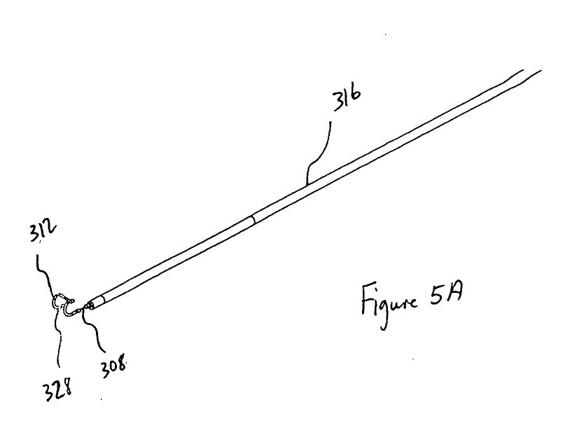

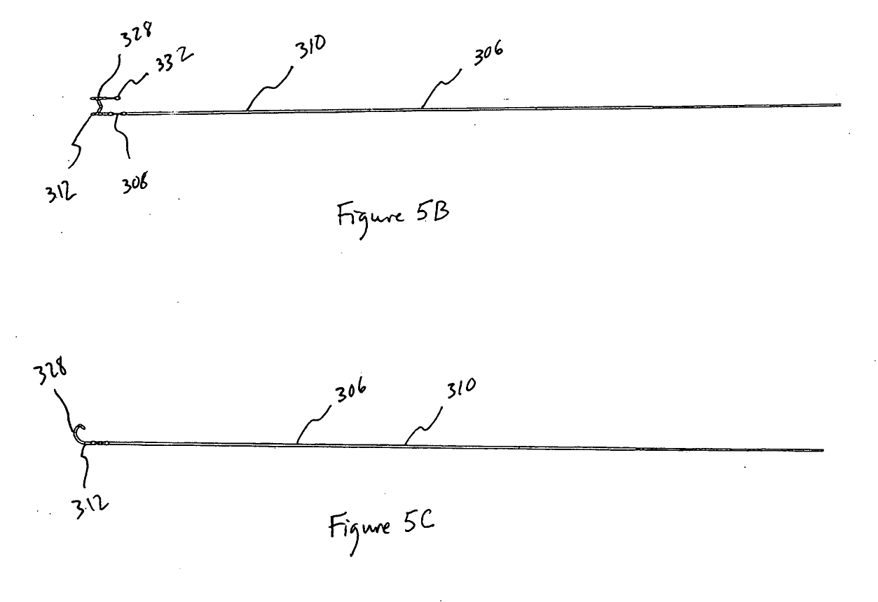

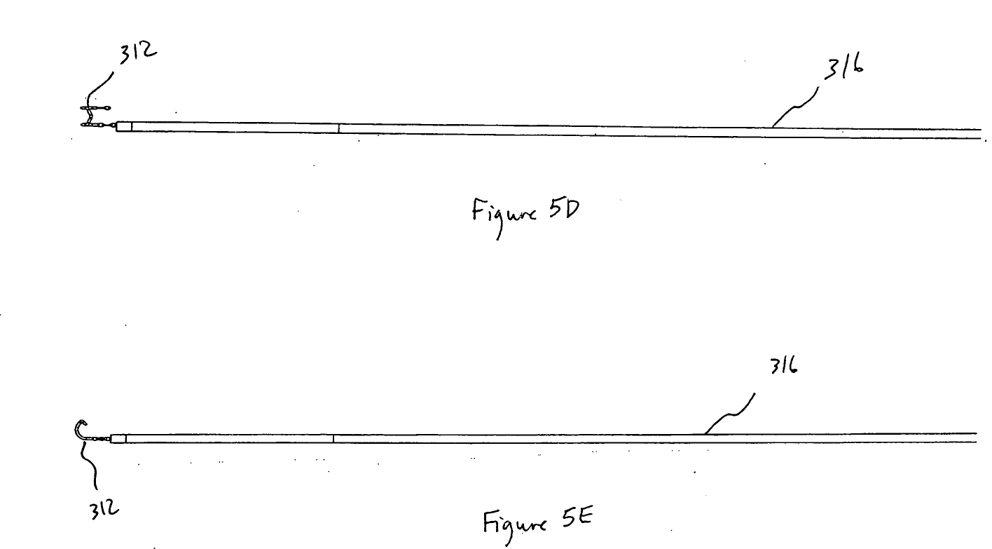

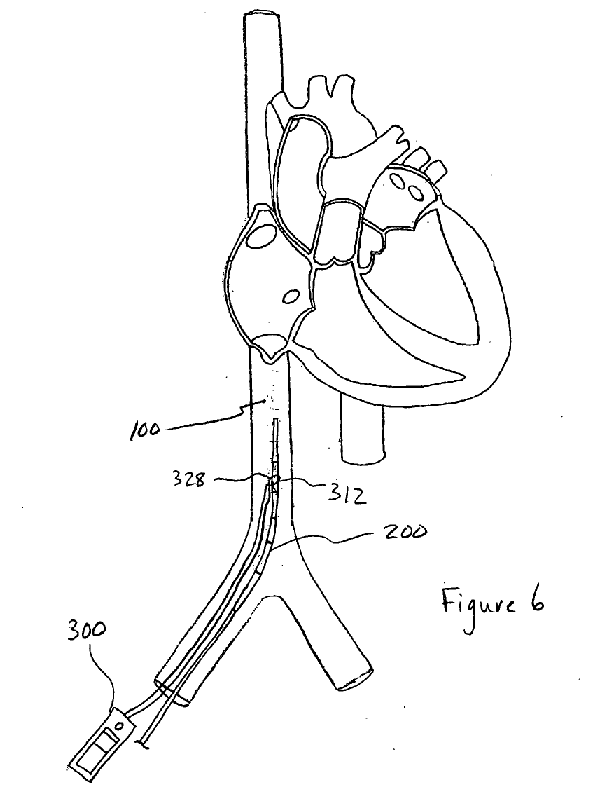

The placement of the implantable intravascular defibrillator (IID) device 200 within a vessel can be performed using a device delivery system 300. Embodiments of a device delivery system 300 are depicted in FIGS. 4A-4E and 5A-5E. Device delivery system 300 can generally include a device body 302 and a handle 304.

The body 302 can include a flexible wire 306. Wire 306 can be a coaxial wire that includes an inner wire 308 and an outer wire 310 that generally surrounds the inner wire 308. A grasper mechanism 312 can be disposed at a distal end of the inner wire 308. Body 302 can also include a flexible sheath 316 surrounding wire 306. The distal end of the flexible sheath 316 can include a collar section 314. The collar section 314 can have an inner diameter sized to incorporate not only the wire 306, but also the grasper mechanism 312. A secondary wire, or stylet, can also be contained in device body. Stylet can be contained within flexible sheath 316 in a separate lumen from wire 306, or can be contained in the same aperture as wire 306 and can be used to form or release a closed loop with grasper mechanism 312.

Handle 304 can define a central longitudinal aperture 318 that can accommodate body 302. A thumb slide 320 can be disposed within an axially positioned slot 322. Thumb slide 320 can be connected to a proximal end of wire 306. Handle 304 can also include a flush port 324. Flush port 324 can be used to flush blood out of delivery device 300 during implantation.

The grasper mechanism 312 has a collar or lasso like shape that permits selective frictional or pressure based grasping and releasing of the IID 200 without the need for a positive mechanical mating of a mandrel as with prior device delivery solutions. The lasso, or lariat, includes a releasable loop 328, or honda, portion. The releasable honda 328 is configured to grasp the IID in order to position the IID 200 in the vasculature. Releasable honda 328 has an end portion 332 that can be connected to the collar section 314 or a stylet to form a closed loop. The grasper mechanism 312 can attach to the proximal end of the IID 200, the distal end of the IID 200, the lead 202, or any other portion of the IID 200 in order to position the IID 200 in the vasculature. In one embodiment, the IID 200 can have a circumferential notch around the device body 204 configured to be grasped by the device delivery system 300. The grasper mechanism 312 can have various shapes. The embodiment shown in FIGS. 4A-4E is generally hook shaped. Another embodiment of a grasper mechanism 312 suitable for device delivery depicted in FIGS. 5A-5E, is generally “w” shaped. Any shape that can be used with the collar section or a stylet to form a closed loop to grasp an IID can be used.

In one embodiment, grasper mechanism 312 can be formed of memory wire. The shape of the grasper mechanism 312 can be formed by setting the memory wire in a heated fixture. Memory wire allows the grasper mechanism to deform when necessary while still naturally retaining the grasping shape.

The thumb slide 320 can move within the axially disposed slot 322 in the handle 304. If the proximal end of inner wire 308 is connected to thumb slide 320, axial movement of the thumb slide 320 produces a corresponding movement of the inner wire 308. A stop fixture 330 can be disposed at the distal end of the slot 322, to prevent the thumb slide 320 from moving past a certain point. The stop fixture 330 reduces the potential for the inadvertent release of the IID 200 during the implantation procedure. Alternatively, the thumb slide 320 can control the operation of a stylet contained within sheath 316 that is separate from wire 306. Alternatively, thumb slide 320 can be switched to operate an extension assembly coupled to outer wire 310, the extension assembly adapted to provide extension of the grasper mechanism.

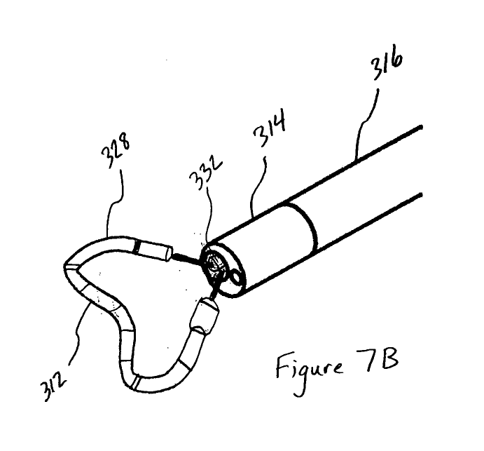

The grasper mechanism 312 can be configured to grasp and release the IID 200 in various ways. In one embodiment, as can be seen in FIGS. 7A and 7B, the end portion 332 of releasable honda 328 of grasper mechanism 312 is manually inserted into collar section 314 of device body 302 to form a closed loop for grasping the IID 200. This can be done outside of the body on the operating table. To release the IID 200, the stop fixture 330 is removed allowing thumb slide 320 to advance into the region previously occupied by the stop fixture 330, pushing the inner wire 308 forward. As the inner wire 308 slides as far forward as it can go, the end portion 332 of releasable honda 328 is pulled out of collar section 314, thereby releasing the IID 200 from the grasp of device delivery system 300. In another embodiment, thumb slide 320 can control a stylet, which can be used to push end portion 332 out of collar section 314.

In another embodiment, a closed loop is formed around an IID 200 by mating a stylet with the end portion 332 of releasable honda 328. To open the loop, the stop fixture 330 can be removed and the thumb slide 320 can move forward to push the inner wire 308 and end portion 332 out of contact with the stylet. Alternatively, the thumb slide 320 can control movement of the stylet and the loop can be opened by withdrawing the stylet out of contact with the end portion 332. In this alternative, it is possible to remotely grasp the IID 200 while it is in or out of the body, by remotely moving the stylet forward with thumb slide 320 to close the loop with the end portion 332 around IID 200.

A distal end of device body 302 of delivery system can also be configured to articulate and/or rotate in order to aid in positioning of an IID 200 or lead 202. Articulation of the distal end of the delivery system can be accomplished through the use of one or more pull wires extending internally from the distal end of the device to the handle. The delivery system may be configured such that rotating the handle with respect to the flexible body pulls the internal wire, causing the articulation of the distal end of lead delivery system. In another embodiment, a supplemental thumb slide may be provided in handle, operably coupled to pull wire to cause articulation of the distal end.

### Implantation Methods

To implant IID 200 with device delivery system 300, first an incision is formed to allow access to the vasculature. In one embodiment, the incision is formed in the femoral vein. An introducer sheath configured to allow insertion of devices into the vasculature while preventing loss of blood can be inserted into the incision to keep the vein open during the procedure.

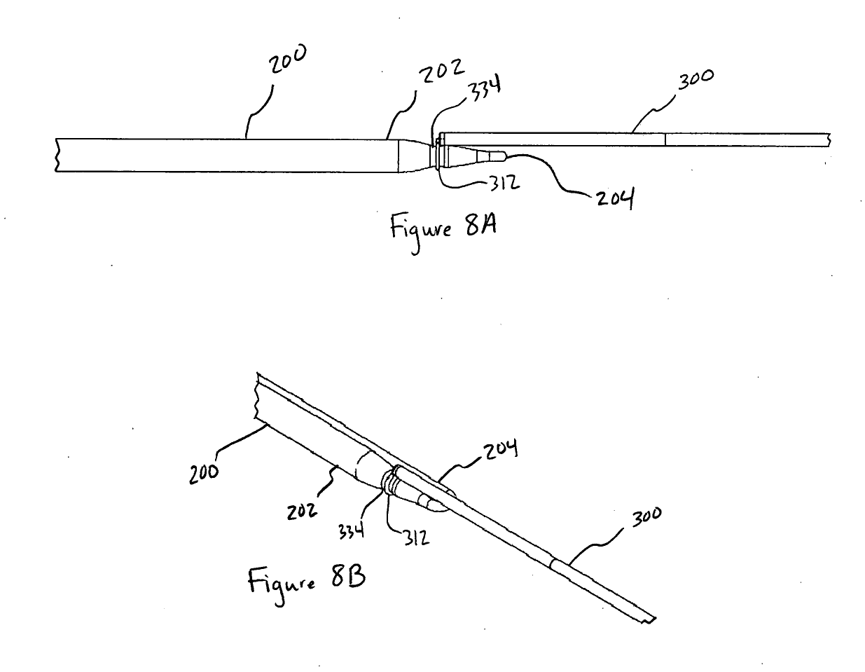

The device delivery system 300 is then used to grasp the IID 200 with the grasper mechanism 312. As described above, the releasable honda 328 of device delivery system 300 can be manually secured around the IID 200 to grasp the IID 200 by various means prior to implantation. The device delivery system 300 can grasp the IID 200 at any point along the IID 200, providing positive control of the IID 200. In one embodiment depicted in FIGS. 8A and 8B, the device delivery system 300 grasps the interface between the device body 204 and the lead 202. IID can be provided with a circumferential notch 334 configured to be grasped by the device delivery system 300.

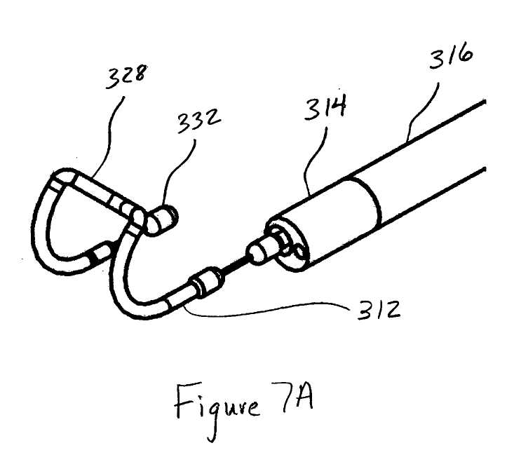

The IID 200 can then be inserted through the introducer sheath. The device delivery system 300 is used to guide the IID 200 through the vasculature to the desired location, as shown, for example, in FIG. 6. In one embodiment, the device delivery system 300 maintains its grasp on the IID 200 until it reaches the desired location. In another embodiment, the device delivery system 300 grasps a portion of the IID 200, for example its distal end 208, for a first part of the implantation process then releases the IID 200 and re-grasps it at a second location, for example its proximal end 206, to complete the implantation process. If at any point during the implantation process the device delivery system 300 becomes overly filled with blood, flush port 324 on handle 304 can be used to flush the blood from the device 300. In some embodiments, fluoroscopy can be utilized to aid in all aspects of the implantation.

In some embodiments, once the IID 200 is positioned in its desired location, the device delivery system 300 can release the IID 200 and be withdrawn from the body. In one embodiment, the IID 200 can be released by removing the lock 330 on handle 304 and operating the thumb slide 320 to open the loop with releasable honda 328. The thumb slide 320 can then be used to completely withdraw the grasper mechanism 312 into the device delivery system 300 device body 302 to prevent it from contacting the patient's body as the device delivery system 300 is removed. In another embodiment, device delivery system 300 can retain position control until after a lead associated with the IID is implanted. Once the IID 200 has been implanted in the desired location and the device delivery system 300 is withdrawn, the IID 200 can be anchored within the body. In one embodiment, the handle 304 remains completely outside of the patient's body during the entire implantation procedure.

In another embodiment implantation of an IID 200 with delivery device 300 can use an over-the-wire technique that provides for additional positive control of the IID 200. An incision can be formed in the femoral vein and an introducer inserted into the incision to keep the vein open during the procedure. A guide catheter is then inserted through the introducer and a guide wire is directed into a vessel superior to the heart across from the target anchor location. The guide catheter is removed leaving the guide wire in place. A distal portion 208 of the IID 200 containing a guidewire passage is inserted over the guide wire, and the IID 200 is manually inserted through the introducer sheath as far as possible. A dissolvable, lubricious coating may be applied to the IID to aid in delivery of the device through the vasculature. A device delivery system 300 then grasps the IID 200 as described above. In one embodiment, the device delivery system 300 grasps the proximal end 206 of the IID 200. In this embodiment, the device delivery system 300 provides positive control of the proximal end 206 of the IID while the guidewire provides positive control of the distal end 204 of the IID 200. The IID 200 is then guided through the vasculature by the device delivery system 300 to the desired location superior of the heart. In one embodiment, the device delivery system 300 is then removed from the vasculature.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A method for positioning an intravascular implantable device in a patient's body, comprising:
forming an incision in the vasculature of the patient;
grasping the intravascular implantable device with a grasper mechanism located at a distal end of a device delivery system by closing a releasable honda around the device;
inserting the intravascular implantable device into the patient's vasculature; and
guiding the intravascular implantable device to a desired location within the patient's body using the device delivery system.

2. The method of claim 1, further comprising:
operating a handle located at a proximal end of the device delivery system to release the grasper mechanism's grasp on the intravascular implantable device, wherein the handle remains outside of the patient's body; and
withdrawing at least a portion of the device delivery system from the patient's vasculature.

3. The method of claim 1, wherein the releasable honda is manually closed around the intravascular implantable device by interfacing an end portion of the releasable honda with a collar section located at a distal end of device delivery system.

4. The method of claim 1, wherein the releasable honda is closed around the intravascular implantable by connecting a stylet with the releasable honda.

5. The method of claim 1, further comprising inserting an introducer sheath into the incision and wherein the intravascular implantable device is inserted into the patient's vasculature through the introducer sheath.

6. The method of claim 2, further comprising removing a stop fixture on handle to allow the grasper mechanism to release its grasp.

7. A method comprising:
providing a device delivery system including a grasper mechanism having a releasable honda disposed at a distal end of device delivery system;
providing instructions for positioning an intravascular implantable device within a patient's vasculature, the instructions comprising:
forming an incision in the vasculature of the patient;
grasping the intravascular implantable device with the grasper mechanism by closing a releasable honda around the device;
inserting the intravascular implantable device into the patient's vasculature; and
guiding the intravascular implantable device to a desired location within the patient's body using the device delivery system.

8. The method of claim 7, wherein the instructions further comprise:
operating a handle located at a proximal end of the device delivery catheter to release the grasper mechanism's grasp on the intravascular implantable device such that the handle remains outside of the patient's body; and
withdrawing at least a portion of the device delivery catheter from the patient's vasculature.

9. The method of claim 7, wherein the releasable honda is manually closed around the intravascular implantable device by interfacing an end portion of the releasable honda with a collar section located at a distal end of device delivery catheter.

10. The method of claim 7, wherein the releasable honda is closed around the intravascular implantable device by connecting a stylet with the releasable honda.

11. The method of claim 7, wherein the instructions further comprise inserting an introducer sheath into the incision and wherein the intravascular implantable device is inserted into the patient's vasculature through the introducer sheath.

12. The method of claim 8, wherein the instructions further comprise removing a stop fixture on handle to allow the grasper mechanism to release its grasp.

13. An apparatus for positioning an intravascular implantable device in a patient's body, comprising:
an elongated flexible device body adapted to be temporarily introduced into the patient's vasculature, the device body having a proximal end and a distal end and including a flexible wire extending through at least a portion of the length of the device body;
a handle operably connected to the proximal end of the device body; and
a grasper mechanism operably connected to a distal end of the flexible wire and configured to releasably grasp an intravascular implantable device having an elongated cylindrical housing, wherein the grasper mechanism is configured to grasp the intravascular implantable device by closing a releasable honda around the cylindrical housing of the device that is selectively controllable with the handle.

14. The apparatus of claim 13, wherein the releasable honda is closed around the intravascular implantable device by interfacing an end portion of the releasable honda with a collar section located at the distal end of the device body.

15. The apparatus of claim 13, wherein the device body further includes a stylet extending through at least a portion of the length of the device body, and wherein the releasable honda is closed around the intravascular implantable device by connecting the stylet with the releasable honda.

16. The apparatus of claim 13, wherein the grasper mechanism is configured to release the intravascular implantable device via operation of the handle.

17. The apparatus of claim 13, wherein the handle includes a slide connected to a proximal end of the flexible wire, and wherein axial movement of the slide relative to the handle produces a corresponding movement of the flexible wire.

18. The apparatus of claim 17, wherein the handle further includes a stop fixture configured to prevent movement of the slide beyond a predetermined position on the handle, and wherein, in response to release of the stop fixture, movement of the handle beyond the predetermined position causes the releasable honda to release its grasp on the intravascular implantable device.

19. The apparatus of claim 13, wherein the device body further includes a flexible sheath surrounding the wire.

20. The apparatus of claim 13, wherein the grasper mechanism can be completely withdrawn into the device body via operation of the handle.

21. An apparatus for positioning an intravascular implantable device in a patient's body, comprising:
an elongated flexible device body adapted to be temporarily introduced into the patient's vasculature, the device body having a proximal end and a distal end and including a flexible wire extending through at least a portion of the length of the device body;
means for grasping an intravascular implantable device having an elongated cylindrical housing by using a releasable honda; and
means for selectively controlling the means for grasping connected to the proximal end of the device body.

22. The apparatus of claim 21, wherein the device body further includes a flexible sheath surrounding the wire.

23. The apparatus of claim 21, wherein the grasper mechanism can be completely withdrawn into the device body via operation of the handle.
